(19) United States
(12) Patent Application Publication
(10) Pub. No.: US 2024/0156918 A1
(43) Pub. Date: May 16, 2024

(54) GEL BASED ON ANNELID HEMOGLOBIN

(71) Applicant: HEMARINA, Morlaix (FR)

(72) Inventors: Franck ZAL, Morlaix (FR); Elisabeth LEIZE-ZAL, Morlaix (FR)

(21) Appl. No.: 18/550,192

(22) PCT Filed: Mar. 14, 2022
(86) PCT No.: PCT/EP2022/056448

§ 371 Date: Sep. 12, 2023

(30) Foreign Application Priority Data
Mar. 15, 2021 (FR) FR2102533

(51) Int. Cl. A61K 38/42; A61K 47/36; A61P 17/02; A61P 31/04

(52) U.S. Cl. CPC A61K 38/42; A61K 47/36; A61P 17/02; A61P 31/04

(57) ABSTRACT

Disclosed is a composition including, in a physiologically acceptable medium: at least one molecule chosen from an Annelid globin, an Annelid globin protomer and an Annelid extracellular hemoglobin; at least one hydrophilic thickening polymer chosen from polymers of natural origin, which are optionally modified, including at least one mannose unit; and hyaluronic acid or a salt thereof.

### CROSS-REFERENCE TO RELATED APPLICATIONS

This application is the US national stage of PCT/EP2022/056448, filed Mar. 14, 2022 and designating the United States, which claims the priority of FR FR2102533, filed Mar. 15, 2021. The entire contents of each foregoing application are incorporated herein by reference.

### BACKGROUND OF THE INVENTION

### Field of the Invention

The present invention relates to a composition comprising at least one molecule chosen from among an Annelid globin, an Annelid globin protomer and an Annelid extracellular hemoglobin, at least one hydrophilic thickening polymer chosen from polymers of natural origin comprising at least one mannose unit, and hyaluronic acid or a salt thereof.

### Description of the Related Art

Periodontal diseases are diseases of infectious origin (bacteria) which affect and destroy teeth-supporting tissues which form the periodontium. The periodontium is formed of four tissues: gingival tissue, alveolar bone, the periodontal ligament, and cementum. When periodontal disease is limited to the gums, the term gingivitis is used. When the entire periodontium is affected, the term used is periodontitis.

Periodontitis is a disease of the epithelial-conjunctive attachment system. It is characterized by loss of attachment i.e. detachment of the junctional epithelium and gingival conjunctive fibers from the tooth surface. This detachment can be pathological corresponding to the formation of a periodontal pocket, or surgical corresponding to a periodontal wound. Periodontal healing therefore consists of reattaching the soft tissues at the tooth surface, one might say of «closing» the periodontal wound.

In its broadest meaning, healing is the mending of a wound i.e. a dynamic process concerning all the tissues of the body and tending to restore the anatomy and function thereof. Periodontal healing has the particular aspect of involving tissues that are biologically distinct, of different type and consistency, unlike the model of skin healing which entails the joining and/or filling of histologically identical soft tissue.

A skin wound is characterized by:
two tissues: the epidermis and the dermis;
a blood clot the volume of which varies as a function of the extent of the wound;
wound edges with greater or lesser separation and under stress.

Conventionally, a distinction is made between healing by primary intention resulting from perfect coaptation of the edges of the wound, and healing by secondary intention resulting from migration of the epidermal cells to fill the distanced wound edges.

Periodontal healing appears to be more complex than skin healing. The same characteristics are found: tissue compartments (epithelium and conjunctive tissue), distance of wound edges, stresses, volume of blood clot.

Nevertheless, this model differs through (1) greater cell diversity associated with the participation of bone and periodontal ligament cells, (2) wound edges of different type and consistency and (3) a specific bacterial environment. Dental plaque (i.e. accumulation of food debris and bacteria) and tartar (i.e. calcification of this dental plaque) adhere to the surface of the tooth underneath the gum line and are colonized by pathogenic bacteria. The stagnation of these bacteria in dental plaque is the cause of an inflammatory reaction on the gums and bone, gradually leading to destruction thereof.

The healing of wounds, whether skin or periodontal, is therefore of major interest for restoring the integrity of the skin barrier.

The current treatment of periodontitis consists of root planing (if necessary, under local anesthesia) to remove the dental plaque and tartar lying under the gum. The objective of this treatment is to cause re-attachment of the previously exposed gum and root surfaces. Periodontal surgery can also be envisaged. However these options remain relatively invasive.

Regarding skin wounds, these can be more or less severe and of different sizes.

There is therefore a need for a treatment of wounds, in particular skin or periodontal wounds, that is simple and efficient and which promotes healing.

In addition, these treatments must be sterile to prevent further contamination of the skin and/or periodontium, but also stable. Finally, they must be of adequate viscosity: not too liquid and therefore able to remain for sufficient time at the site of action (for example in a periodontal pocket or on a skin wound) and not too solid so that they can be relatively easily resorbed.

With the present invention, it is possible to meet these expectations.

### SUMMARY OF THE INVENTION

Surprisingly, the Applicant has found that the formulation of at least one molecule chosen from among an Annelid globin, an Annelid globin protomer and an Annelid extracellular hemoglobin, in a gel comprising a hydrophilic thickening polymer of natural origin having at least one mannose unit and hyaluronic acid or a salt thereof, allows the obtaining of a stable efficient composition in particular for healing and in particular of skin or periodontal wounds. In addition, said composition allows the reliable administering of the molecule in situ and with satisfactory length of action time (i.e. at least a few hours, preferably at least 5 hours, preferably at least 10 hours, preferably at least 12 hours). Finally, said composition is typically sterile and has the required viscosity. Finally, said composition is biocompatible, biodegradable, and resorbable.

The subject of the present invention is a composition («composition of the invention») comprising, in a physiologically acceptable aqueous medium:
at least one molecule chosen from among an Annelid globin, an Annelid globin protomer and an Annelid extracellular hemoglobin;
at least one hydrophilic thickening polymer chosen from among polymers of natural origin, optionally modified, comprising at least one mannose unit, and
hyaluronic acid or a salt thereof.

The invention also relates to the use of a composition of the invention as medicinal product.

Preferably, the invention relates to the use of a composition of the invention to prevent and/or treat periodontal disease, and/or for the treatment of at least one periodontal pocket, and/or to promote healing of the skin and/or periodontium, and/or to treat (i.e. promote and/or increase) bone healing.

Preferably, the invention relates to the use of a composition of the invention to prevent and/or treat a skin disease, preferably acne.

Preferably, the composition of the invention has rheofluidifying properties.

These properties can be evaluated with the following protocol: a rheometer is used (Thermo Scientific Haake Mars, modular advanced rheometer system) with parallel plate geometry (diameter 40 mm, gap 1 mm). A suitable amount of sample is loaded on a Peltier plate equipped with a temperature control system for efficient, accurate thermoregulation (±0.001° C.). The viscosity flow rate curves are measured with a constant velocity sweep method (0.1 to 10 000 Pa·s). The Linear Viscoelastic Range—LVER) of the sample is determined by dynamic amplitude sweep and with viscoelastic modules (G′ storage modulus and G″ loss modulus) and by frequency sweep testing. In these tests, continuous excitation is applied to the sample over the LVER range to avoid destroying the sample. The amplitude sweep test range is between 0.01 and 100% and at an angular frequency of 0.1 Hz at 20° C. for both tests. Frequency sweep analysis is performed on the sample in the rheometer at 20° C., over the angular frequency range of between 0.1 and 100 Hz at a constant stress of 1%.

The composition of the invention has elastic properties. In addition, G′ remains higher than G″ even at low frequency.

The composition of the invention comprises at least one molecule chosen from among an Annelid globin, an Annelid globin protomer and an Annelid extracellular hemoglobin.

This molecule is an oxygen carrier. By «oxygen carrier», it is meant a molecule capable of reversibly transporting oxygen from the environment to cells, tissues, or target organs.

Annelid extracellular hemoglobin is contained in the three classes of Annelids: Polychaeta, Oligochaeta and Hirudinea. The term extracellular hemoglobin is used since it is naturally not contained in a cell, and it is therefore able to circulate freely within the blood system without chemical modification for stabilization or functionalization thereof.

Annelid extracellular hemoglobin is a giant biopolymer having a molecular weight of between 2000 and 4000 kDa, formed of about 200 polypeptide chains of between 4 and 12 different types that are generally grouped into two categories.

The first category comprising 144 to 192 elements, groups together the so-called “functional” polypeptide chains which carry an active site of heme type and are capable of reversibly binding to oxygen; they are chains of globin type (a total of eight types for the hemoglobin of Arenicola marina: a1, a2, b1, b2, b3, c, d1 and d2), having molecular weights of between 15 and 18 kDa. They are very similar to the chains of type α and β of vertebrates.

The second category, having 36 to 42 elements, groups together the so-called «structure» polypeptide chains or «linkers» having no or scarcely no active site but allowing the assembling of subunits called twelfths or protomers. There are two types of linkers, L1 and L2.

Each hemoglobin molecule is composed of two superimposed hexagons called a hexagonal bilayer, and each hexagon is itself formed by the assembly of six subunits (dodecamer or protomer) in the shape of a water drop. The native molecule is formed of twelve of these subunits (dodecamer or protomer). Each subunit has molecular weight of about 250 kDa, and forms the functional unit of the native molecule.

Preferably, the Annelid extracellular hemoglobin is chosen from among extracellular hemoglobins of Polychaeta Annelids and extracellular hemoglobins of Oligochaeta Annelids. Preferably, the Annelid extracellular hemoglobin is chosen from among extracellular hemoglobins of the Lumbricidae family, extracellular hemoglobins of the Arenicolidae family and extracellular hemoglobins of the Nereididae family. More preferably, the Annelid extracellular hemoglobin is chosen from among the extracellular hemoglobin of Lumbricus terrestris, the extracellular hemoglobin of Arenicola sp and the extracellular hemoglobin of Nereis sp. Further preferably according to the invention, the extracellular hemoglobin of Arenicola marina or Nereis virens, still further preferably the extracellular hemoglobin of Arenicola marina. The Arenicola marina lugworm or sandworm is a polychaeta Annelid worm essentially living in the sand.

In the invention, the globin protomer of Annelid extracellular hemoglobin forms the functional unit of native hemoglobin, as indicated above.

Finally, the globin chain of Annelid extracellular hemoglobin can particularly be chosen from among globin chains of type Ax and/or Bx of Annelid extracellular hemoglobin.

Annelid extracellular hemoglobin, the globin protomers and/or globins thereof do not require a co-factor for functioning, contrary to mammalian and especially human hemoglobin. Finally, since Annelid extracellular hemoglobin, the globin protomers and/or globins thereof do not have any blood typing, they avoid any problems of immunological or allergic reaction. Annelid extracellular hemoglobin, the globin protomers and/or globins thereof have intrinsic superoxide dismutase action (SOD). As a result, this intrinsic antioxidant action does not require any antioxidant for functioning unlike the use of a mammalian hemoglobin for which the antioxidant molecules are contained inside the red blood cell and are not bound to the hemoglobin.

Annelid extracellular hemoglobin, the globin protomers and/or globins thereof can be native or recombinant.

Preferably, the extracellular hemoglobin is that of Arenicola marina or of Nereis virens, more preferably the extracellular hemoglobin of Arenicola marina.

Preferably, the molecule is contained in the composition of the invention in an amount of between 0.01% and 10% by weight relative to the total weight of the composition, preferably between 0.05% and 5% by weight, preferably between 0.06% and 2% by weight, preferably between 0.07% and 1% by weight, preferably between 0.08% and 0.5% by weight, preferably between 0.09% and 0.3% by weight.

Preferably, the molecule chosen from among an Annelid globin, an Annelid globin protomer and an Annelid extracellular hemoglobin of the invention is formulated in a buffer solution. The solution obtained (i.e. buffer solution comprising the molecule) can be lyophilized to obtain a powder. Preferably, the solution obtained (i.e. buffer solution comprising the molecule) is used as such (liquid form) in non-lyophilized form.

Typically, the buffer solution comprising the lyophilized or non-lyophilized molecule (and in this case liquid molecule) is added to the mixture comprising the hydrophilic thickening polymer and hyaluronic acid, to obtain the composition of the invention.

The buffer solution creates a suitable saline environment for the hemoglobin, the protomers and globins thereof, and thereby allows maintaining of the quaternary structure and hence of the functionality of this molecule. The buffer solution is preferably an aqueous solution comprising salts, preferably sodium, calcium, magnesium, and potassium chloride ions, with a pH of between 5 and 9, preferably between 5.5 and 8.5, preferably between 6.5 and 7.6. The formulation thereof is similar to that of a physiologically injectable liquid. Preferably, the buffer solution also comprises an antioxidant such as ascorbic acid. Under these conditions, the Annelid extracellular hemoglobin, the globin protomers and globins thereof remain functional.

In the present description, pH is to be understood at ambient temperature (25° C.), unless otherwise stated. Preferably, the buffer solution is an aqueous solution comprising sodium chloride, calcium chloride, magnesium chloride, potassium chloride, together with sodium gluconate and sodium acetate, and has a pH of between 6.5 and 7.6, preferably of 7.1±0.5, preferably of about 7.35. More preferably, the buffer solution is an aqueous solution comprising 90 mM NaCl, 23 mM Na-gluconate, 2.5 mM CaCl₂), 27 mM Na-acetate, 1.5 mM MgCl2, 5 mM KCl, and has a pH of 7.1±0.5.

The composition of the invention also comprises at least one hydrophilic thickening polymer chosen from among polymers of natural origin having at least one mannose unit. These polymers of natural origin can be modified, for example through the addition of one or more hydroxypropyl groups, the addition of groups of methylcarboxylate salts such as sodium methylcarboxylate, or the addition of trimethyl ammonium groups.

Preferably, the hydrophilic thickening polymer is a polymer of non-modified natural origin comprising at least one mannose unit.

Preferably, the hydrophilic thickening polymer is chosen from among:
xanthan gum and derivatives of xanthan;
glucomannans and derivatives thereof, such as konjac gum;
galactomannans and derivatives thereof, such as locust bean gum, fenugreek gum, tara gum, guar gum or derivatives of guar gum such as hydroxypropylguar, hydroxypropylguar modified by sodium methylcarboxylate groups (such as the product sold under the trade name Jaguar XC97-1 by Rhodia) or guar hydroxypropyl trimethyl ammonium chloride;
and mixtures thereof.

Preferably, the hydrophilic thickening polymer is chosen from among:
xanthan gum;
konjac gum;
locust bean gum, fenugreek gum, tara gum, guar gum;
and mixtures thereof.

Preferably, the hydrophilic thickening polymer is xanthan gum.

Xanthan gum is an anionic polysaccharide of high molecular weight (about 10⁶) produced by fermentation of carbohydrates by Xanthomonas campestris. It is composed of a main chain formed of D-glucose units connected by β(1->4) glucosidic bonds; every second anhydroglucose unit carries a trisaccharide side chain composed of a glucuronic acid residue between 2 mannose units. Most of the end units contain a pyruvate group and the mannose unit adjacent to the main chain can be acetylated to C6.

For example, xanthan gum can be sold by Cargill under the trade name Satiaxane UCX 930 or Satiaxane UCX 911.

Preferably, the hydrophilic thickening polymer is contained in an amount of between 0.5% and 5% by weight relative to the total weight of the composition, preferably between 0.8% and 4% by weight, preferably between 1% and 3% by weight, preferably between 1.5% and 2.5% by weight.

The composition of the invention also comprises hyaluronic acid or a salt thereof.

Hyaluronic acid is a disaccharide polymer, in particular a glycosaminoglycan, formed of D-glucuronic acid and N-acetyl-D-glucosamine.

It is naturally present in numerous tissues, chiefly in the skin and in particular in the epidermis, and in conjunctive tissue and represents one of the main constituents of the extracellular matrix. The length of the molecule varies according to tissue, species, and the condition of the tissue.

Hyaluronic acid can be obtained by tissular extraction from animal tissues or via bacterial fermentation in particular with Streptococcus equi or Bacillus subtilis.

Preferably, the hyaluronic acid of the invention is obtained by bacterial fermentation, in particular with Streptococcus equi or Bacillus subtilis, more particularly with Streptococcus equi.

Preferably, the composition of the invention comprises a salt of hyaluronic acid, called hyaluronate. Preferably the composition of the invention comprises a sodium salt (sodium hyaluronate). Preferably the hyaluronic acid or a salt thereof is non-sulfated.

Preferably, the composition of the invention comprises hyaluronic acid or a salt thereof in a content of at least 0.1% by weight, preferably at least 0.2% by weight, preferably at least 0.3% by weight, preferably at least 0.4% by weight, preferably at least 0.5% by weight relative to the total weight of the composition.

Preferably, the hyaluronic acid or a salt thereof is contained in an amount of between 0.3% and 5% by weight relative to the total weight of the composition, preferably between 0.5% and 3% by weight, preferably between 0.8% and 2% by weight.

The hyaluronic acid or a salt thereof can be a hyaluronic acid or hyaluronate of low molecular weight, a hyaluronic acid or hyaluronate of high molecular weight, or a mixture of both.

The hyaluronic acid or hyaluronate of high molecular weight can have a molecular weight ranging from 5 to 5000 kDa, in particular from 6 to 4800 kDa, more particularly from 8 to 4500 kDa (4.5 MDa). Preferably the hyaluronic acid or hyaluronate of high molecular weight has a molecular weight ranging from 1400 to 4000 kDa, preferably from 1500 to 3500 kDa, preferably from 1500 to 3400 kDa.

The hyaluronic acid or hyaluronate of low molecular weight can have a molecular weight ranging from 10 to 1000 kDa, in particular from 10 to 600 kDa.

Molecular weight can be measured with the conventional method of HPLC elution/exclusion.

In one particular embodiment, the composition as hyaluronic acid or a salt thereof solely comprises hyaluronic acid or a hyaluronate of high molecular weight. This particularly means that the weight ratio of hyaluronic acid (or hyaluronate) of low molecular weight relative to the hyaluronic acid (or hyaluronate) of high molecular weight is less than or equal to 0.1%, in particular less than 0.01%, and is even 0.

In particular, the composition has a content of high molecular weight hyaluronic acid (or hyaluronate) of between 0.3% and 5% by weight relative to the total weight of the composition, preferably between 0.5% and 3% by weight, preferably between 0.8% and 2% by weight.

Preferably, the hyaluronic acid or a salt thereof has intrinsic viscosity at 25° C. ranging from 1 to 4 m³/kg, preferably from 1.4 to 3.8 m³/kg, preferably from 1.7 to 3.4 m³/kg, preferably from 2 to 3.4 m³/kg, preferably from 2.5 to 3.4 m³/kg. The calculation of intrinsic viscosity is a parameter well known to persons skilled in the art and can be carried out as indicated in the European Pharmacopeia (European Pharmacopeia 9.0, Monographs S, Sodium (hyaluronate), pages 3834-3835).

Preferably the composition of the invention, in a physiologically acceptable aqueous medium, comprises:
an extracellular hemoglobin of Arenicola marina or Nereis virens, preferably in an amount of between 0.05% and 0.5% by weight relative to the total weight of the composition, preferably between 0.08% and 0.4% by weight, preferably between 0.09 and 0.3% by weight;
xanthan gum, preferably in an amount of between 0.5% and 5% by weight relative to the total weight of the composition, preferably between 1% and 3% by weight, preferably between 1.5% and 2.5% by weight; and
sodium hyaluronate, preferably of high molecular weight, preferably in an amount of between 0.4% and 5% by weight relative to the total weight of the composition, preferably between 0.5% and 4% by weight, preferably between 0.8% and 2% by weight.

Preferably, the composition of the invention, in a physiologically acceptable aqueous medium, comprises:
an extracellular hemoglobin of Arenicola marina, in an amount of between 0.09 and 0.3% by weight;
xanthan gum in an amount of between 1.5% and 2.5% by weight; and
sodium hyaluronate, preferably having intrinsic viscosity at 25° C. ranging from 1 to 4 m³/kg, preferably from 1.4 to 3.8 m³/kg, preferably from 1.7 to 3.4 m³/kg, preferably from 2 to 3.4 m³/kg, preferably from 2.5 to 3.4 m³/kg, in an amount of between 0.8% and 2% by weight.

The composition of the invention also comprises a physiologically acceptable aqueous medium. By «physiologically acceptable», it is meant that the medium is compatible with application to the skin and/or into the periodontal pocket. Preferably, said medium is sterile.

The medium typically comprises water. Preferably the amount of water is at least 80% by weight, preferably at least 90% by weight, preferably at least 95% by weight relative to the total weight of the composition.

Preferably, the composition of the invention is a gel.

Preferably, the composition of the invention is administered via injection or via in situ instillation into the area to be treated. Preferably, in particular for periodontal pathologies, the composition of the invention is administered via local route, via injection or in situ instillation into the hollow of the periodontal pocket or onto the wound. Preferably, the composition of the invention is administered via instillation directly onto the area to be treated, typically into the hollow of the periodontal pocket or onto the wound to be treated. It can also be administered at the bone of the periodontium, directly onto the bone or adjacent thereto.

Preferably, the composition of the invention is used in therapy, preferably for the prevention and/or treatment of periodontal disease, and/or to treat at least one periodontal pocket, and/or to promote healing of the skin and/or of the periodontium. Preferably, the composition of the invention is used to prevent and/or treat periodontal disease, by promoting reattachment between the gum and the surface of teeth roots, but also by promoting healing of bone tissue.

Preferably, the composition of the invention is used to treat (i.e. promote and/or increase) bone healing.

Preferably, composition of the invention is used as anti-inflammatory, in particular to treat inflammation caused by hypoxia, and/or to inhibit degradation of tissues caused by P. gingivalis.

As explained in the examples, the anti-inflammatory action of the composition of the invention allows the prevention and/or treatment of periodontal disease or of a periodontal pocket: by inhibiting inflammation caused by hypoxia in this type of pathology, the composition of the invention can slow the progress of periodontitis.

In addition, the composition of the invention inhibits degradation of tissue caused by P. gingivalis, thereby promoting healing thereof.

Preferably, the composition of the invention is used to prevent and/or treat a skin disease, preferably an inflammatory skin disease, preferably acne. By «inflammatory skin disease», it is meant any skin disorder accompanied by an inflammatory component. The term notably includes rosacea, acne, eczema, hand eczema, urticaria, facial erythema, erythema pudicitiae, pruritus, atopic dermatitis, and psoriasis in all forms thereof such as cutaneous, mucosal or ungual, or psoriatic arthritis.

The composition of the intention is able to inhibit inflammation caused by bacteria involved in these pathologies; skin diseases such as acne involve a bacterial and inflammatory component. As member of the resident human microbiota, the Gram positive anerobic bacterium Propionibacterium acnes (P. acnes, now called Cutibacterium acnes) is essentially found in the sebaceous gland of the skin. P. acnes has an estimated skin density of 10² to 10⁵⁻⁶ cm⁻²; it is a well-known opportunistic pathogen. Sometimes this bacterium, which normally lives on the skin surface, causes an inflammation (but not infection) of the hair follicles. If the inflammation develops close to the skin surface, red pimples, or yellow pimples (pustules) can form. Deeper inflamed lesions (nodules and cysts) can form if the infection is closer to hair roots. In very severe acne, the cysts can group together to form even larger and deeper inflamed lesions (acne conglobata), but this is rare. P. acnes and the lesions can also be secondarily infected with Staphyloccocus aureus.

Preferably, the acne is chosen from among acne vulgaris, comedonal acne, polymorphic acne, acne caused by rosacea, nodulocystic acne, acne conglobata and senile acne.

The invention also relates to a device comprising:
a syringe, and
a composition of the invention.

The syringe contains the composition of the invention.

In the device of the invention, the syringe can be connected to a hollow needle, preferably a hollow needle provided with a side hole. Typically, said needle may or may not be present when administering the composition of the invention into the hollow of a periodontal pocket; if not present, in this case the composition is delivered directly from the syringe. Preferably, the composition of the invention is applied to the periodontal pocket or any cavity defect with a hollow needle, preferably a hollow needle having a side hole, preferably with a rounded tip. Preferably, the composition of the invention is applied to the skin (cutaneous application) directly from the syringe.

### DETAILED DESCRIPTION

The invention is illustrated with the following examples.

### Example 1: Preparation of a Composition of the Invention

Under sterile conditions, the following composition was prepared:

TABLE 1

| Ingredient | Quantity |
|---|---|
| Extracellular | 1 g/l |
| hemoglobin of | |
| Arenicola marina | |
| in a buffer | |
| solution | |
| (Hemarina) | |
| Xanthan gum | 2 |
| | (wt. % relative to |
| | total weight of |
| | the composition) |
| Sodium | 1 |
| hyaluronate | (wt. % relative to |
| (Sodium | total weight of |
| Hyaluronate by | the composition) |
| HTL or Contipro) | |
| Buffer | q.s. 100 |
| | (wt. % relative to |
| | total weight of |
| | the composition) |

All the ingredients were mixed in a buffer until a gel was obtained. The composition is in the form of a gel.

Once prepared, the composition was packaged in 1 ml syringes. The syringes were frozen.

The stability thereof was evaluated.

It was found that the composition is stable for at least 3 months at 4° C., and that the hemoglobin of the composition is also stable and functional.

### Example 2: Effect of the Composition of the Invention on Healing

### 1. Aim of the Study

The aim of this study was to evaluate the efficacy and safety of healing products, including the gel of the invention, for wound healing in mini-pigs.

The study lasted 22 days, and was halted on healing of most of the wounds.

It included 3 mini-pigs from Ellegaard: M1, M2 and M3 (M1 and M2 were males, and M3 a female).

The animals were accommodated following standard procedures.

### 2. Study Design

2.1. Treatment Products

The design is explained in the Table below.

TABLE 2

| Group | Name | Description |
|---|---|---|
| 1 | Hydrocellular | Ready-to-use dressing |
| | dressing (Urgo | |
| | Tul Border) | |
| 2 | 0.9% NaCl | Ready-to-use solution for |
| | | injection |
| 3 | Gel K-Y | Sterile lubricant that is non- |
| | lubricating | greasy (based on propylene |
| | jelly (Reckitt | oxide and chlorhexidine |
| | Benckiser | gluconate in a matrix of |
| | Healthcare) | plant mucilage, glycerine |
| | | and water) |
| 4 | Gel of the | Composition of the invention |
| | invention | packaged in 1 ml syringe |
| | (Hemarina) | (such as prepared in Example |
| | | 1) |

2.2. Treatment Plan

Treatment was given on the day of wound creation (D1) and at each change of dressing i.e.: D1, D3, D5, D8, D10, D12, D15, D17 and D19.

The wound was cleaned before applying the product:
Hydrocellular dressing: the dressing was applied directly onto the wound to replace the Mepore® dressing (Mölnlycke, 6×7 cm);
0.9% NaCl: the absorbent portion of the Mepore® dressing was soaked with 0.9% NaCl 0.9% before applying the dressing;
Gel K-Y lubricating jelly: the product was applied directly onto the wound before applying the Mepore® dressing;
Gel of the invention: the product was applied directly onto the wound before applying the Mepore® dressing.

2.3. Forming of Wounds

Two types of full-thickness wounds were made: round using a biopsy punch, and square using a scalpel. Eight (8) wounds were made per animal (4 round/20 mm in diameter and 4 square/sides of 20 mm), allowing 4 treatments, each treatment being represented per type of wound).

2.3. Macroscopic Evaluation of the Wounds

Macroscopic evaluation of the wounds was performed with the following parameters:

A macroscopic evaluation was carried out on each change of treatment/dressing (documented by photos and score grades).

### 3. Results

3.1. General Observation

Results are positive: the gel of the invention packaged in a syringe, is easy to use and apply, the product remains well in place in the wound during treatment.

3.2. Macroscopic Evaluation of the Wounds

The macroscopic observations were as expected for this type of study.

The first signs of epithelialization occurred on and after D5 for the K-Y gel, the gel of the invention and the hydrocellular dressing, and on and after D8 for NaCl.

Local reactions around the wound (erythema, edema, and induration) were limited and mainly concerned the first days of the study; they can be accounted for by excess application of product (erythema for the K-Y gel or gel of the invention). There was no local reaction around the wound with NaCl.

Regarding the overall healing of the wound, it can be noted that on completion of the study (D22) all the wounds treated with the gel of the invention were completely healed, while with the K-Y gel and NaCl one wound had not yet healed, and with the hydrocellular dressing all the wounds had not yet completely healed.

It was also reported that the treatment with the gel of the invention caused far fewer scabs than with the other treatments.

The gel of the invention therefore exhibits very good healing properties.

### Example 3: Effect of the Composition of the Invention on Periodontal Disease

Objectives: Periodontitis is characterized by deep periodontal pockets associated with dysbiotic flora and a hypoxic microenvironment, which exacerbates inflammation and degradation of tissues.

The extracellular hemoglobin of Arenicola marina has excellent oxygen-carrying potential and antioxidant capability, and has recently demonstrated anti-inflammatory and antibacterial properties.

The purpose of this study was to evaluate the role of this hemoglobin in reducing hypoxia and oxidative stress in vitro and in vivo.

Methods: Oral epithelial cells in 2D and 3 D culture were infected with P. gingivalis (MOI=100) or exposed to cobalt chloride to induce hypoxia and oxidative stress, and treated with the hemoglobin (1 g/L) for 24 h.

Hypoxia was evaluated by fluorescence microscopy, and quantification of the expression of the key markers of hypoxia (subunit a of hypoxia-inducible factor (HIF-1α), glucose-1 transporter (Glut-1) and glucose-3 transporter (Glut-3)) were evaluated by RT-qPCR and Elisa.

In vivo, experimental periodontitis was induced by placing ligature threads soaked with P. gingivalis for 3 weeks, and the lesions were treated by applying the gel of the invention.

Healing was evaluated by histomorphometric analysis and TRAP staining after 2 weeks.

Results: In culture, treatment with the gel of the invention reduced hypoxia and oxidative stress induced by P. gingivalis and cobalt chloride. After 24 h treatment, the gene expression relating to the key markers of hypoxia induced by P. gingivalis (HIF-1α, Glut-1, Glut-3) was decreased by 85%, 42% and 83% respectively.

In vivo, histomorphometric analyses showed a reduction in the inflammation score and an improvement in clinical attachment in mice treated with the gel of the invention compared with the control (p<0.05), and reduced osteoclastic activity. Immunohistochemical analysis also showed a decrease in the expression of HIF-1α in soft tissues in the group treated with the gel of the invention.

Conclusions: The extracellular hemoglobin of Arenicola marina is a highly promising molecule capable of improving inflammation induced by hypoxia and tissue degradation induced by P. gingivalis.

As a result, it exhibits major therapeutic potential for the management of periodontitis.

### Example 4: Comparative Tests

All the percentages are weight percentages relative to the total weight of the composition.

1/The following comparative compositions were prepared by mixing the ingredients:

They are compared with the formula of the invention in Example 1.

TABLE 3

| | Composition | |
|---|---|---|
| | HA | XG |
| | Ingredient | |
| | Quantity | Quantity |
| Extracellular | 1 g/l | 1 g/l |
| hemoglobin of | | |
| Arenicola marina | | |
| in a buffer | | |
| solution | | |
| (Hemarina) | | |
| Xanthan gum | — | 3 |
| | | (wt. % relative |
| | | to total |
| | | weight of the |
| | | composition) |
| Sodium | 1 | — |
| hyaluronate | (wt. % relative | |
| (Sodium | to total | |
| Hyaluronate by | weight of the | |
| HTL, intrinsic | composition) | |
| viscosity of | | |
| 3.07 m³/kg) | | |
| Buffer | q.s. 100 | q.s. 100 |
| | (wt. % relative | (wt. % relative |
| | to total | to total |
| | weight of the | weight of the |
| | composition) | composition) |

The stability of the viscosity of composition HA was evaluated at 37° C.+/−2° C. at T0 and at T+12 h.

Measurements of viscosity were recorded at the Newtonian plateau at 37° C.+/−2° C. for a shear rate of 0.01 s⁻¹. This measurement was measurement at T0. After measurement at T0, the gels were placed over a hot water bath at 37° C.+/−2° C. for 12 h. Measurements of viscosity were then recorded at the Newtonian plateau at 37° C.+/−2° C. for a shear rate of 0.01 s⁻¹. This measurement was measurement at T+12 h.

Composition HA, at 37° C.+/−2° C., did not exhibit stable viscosity throughout the minimum time required for use.
The transfer rate of dioxygen between two compartments separated by media of different composition (i.e. scarcely mineralized water with O2 saturation, versus scarcely mineralized water with O2 desaturation) was measured for composition XG. The results show that this composition XG exhibits a lower transfer rate than the composition in Example 1 (data not shown).
The composition in Example 1 of the invention exhibits adequate viscosity, and a dioxygen transfer rate higher than composition XG.

These results show that solely the composition of the invention allows the obtaining of a stable composition, having adequate viscosity and allowing good diffusion of dioxygen.

1. A composition, in a physiologically acceptable aqueous medium, comprising:
at least one molecule chosen from an Annelid globin, an Annelid globin protomer and an Annelid extracellular hemoglobin;
at least one hydrophilic thickening polymer chosen from polymers of natural origin comprising at least one mannose unit; and
hyaluronic acid or a salt thereof.

2. The composition according to claim 1, wherein the molecule is chosen from among the extracellular hemoglobins of the Lumbricidae family, the extracellular hemoglobins of the Arenicolidae family, and the extracellular hemoglobins of the Nereididae family.

3. The composition according to claim 1, wherein the hydrophilic thickening polymer is chosen from among xanthan gum and the derivatives of xanthan; glucomannans and derivatives thereof; galactomannans and derivatives thereof; and mixtures thereof.

4. The composition according to claim 1, wherein the hydrophilic thickening polymer is chosen from among xanthan gum and the derivatives of xanthan.

5. The composition according to claim 1, wherein the hyaluronic acid or a salt thereof is obtained by bacterial fermentation.

6. The composition according to claim 1, wherein the hyaluronic acid or a salt thereof is sodium hyaluronate.

7. The composition according to claim 1, wherein the hyaluronic acid or a salt thereof has a molecular weight ranging from 1400 to 4000 kDa.

8. The composition according to claim 1, wherein the hyaluronic acid or a salt thereof has intrinsic viscosity at 25° C. ranging from 1 to 4 m³/kg.

9. The composition according to claim 1, wherein the molecule is contained in an amount of between 0.01% and 10% by weight relative to the total weight of the composition, preferably between 0.05% and 5% by weight.

10. The composition according to claim 1, wherein the hydrophilic thickening polymer is contained in an amount of between 0.5% and 5% by weight relative to the total weight of the composition; and/or the hyaluronic acid or a salt thereof is contained in an amount of between 0.3% and 5% by weight relative to the total weight of the composition.

11. The composition according to claim 1, comprising:
an extracellular hemoglobin of Arenicola marina or of Nereis virens;
xanthan gum; and
sodium hyaluronate.

12. The composition according to claim 1, comprising:
an extracellular hemoglobin of Arenicola marina, in an amount of between 0.09% and 0.3% by weight;
xanthan gum in an amount of between 1.5% and 2.5% by weight; and
sodium hyaluronate in an amount of between 0.8% and 2% by weight.

13. A device comprising:
a syringe; and
a composition according to claim 1.

14. A method for treating a subject in need thereof, comprising administering to said subject the composition according to claim 1.

15. A method for preventing and/or treating a periodontal disease, and/or for treating at least one periodontal pocket, and/or for promoting healing of the skin and/or of the periodontium, and/or for treating bone healing, and/or for preventing and/or treating a skin disease in a subject, comprising administering to said subject the composition according to claim 1.
